(19) United States
(12) Patent Application Publication
(10) Pub. No.: US 2019/0203269 A1
(43) Pub. Date: Jul. 4, 2019

(54) TRI-NUCLEOTIDE ROLLING CIRCLE AMPLIFICATION

(71) Applicant: UNIVERSITY OF MIAMI, Miami, FL (US)

(72) Inventors: Jean-Marc Zingg, Miami, FL (US); Sylvia Daunert, Miami, FL (US)

(21) Appl. No.: 16/331,099

(22) PCT Filed: Sep. 6, 2017
(86) PCT No.: PCT/US17/50292

§ 371 Date: Mar. 6, 2019

Related U.S. Application Data
(60) Provisional application No. 62/384,470, filed on Sep. 7, 2016.

(51) Int. Cl. C12Q 1/6809; C12Q 1/6844; C12Q 1/6876

(52) U.S. Cl. CPC C12Q 1/6809; C12Q 2531/125; C12Q 1/6876; C12Q 1/6844

(57) ABSTRACT

Kits and methods for detecting a target polynucleotide sequence are disclosed. The kits and methods described herein allow for the detection of a target polynucleotide sequence that lacks a missing base selected from adenine, cytosine, guanine, thymine, uracil, and combinations thereof, using rolling circle amplification and a padlock probe polynucleotide sequence that lacks the base complementary to the base missing in the target polynucleotide sequence. The kits and methods may be used to detect any target polynucleotide sequence, such as DNA or RNA from a bacterial, fungal, or viral pathogen.

### CROSS-REFERENCE TO RELATED APPLICATION AND INCORPORATION BY REFERENCE

This application claims the benefit of priority under 35 U.S.C. § 119(e) of U.S. Provisional Patent Application No. 62/384,470, filed Sep. 7, 2016, which is incorporated herein by reference.

This application contains, as a separate part of the disclosure, a sequence listing in computer-readable form (Filename:51002A_Seqlisting.txt; Size:7,111 bytes; Created: Sep. 6, 2017), which is incorporated by reference in its entirety.

### FIELD OF THE INVENTION

The disclosure relates to methods and kits for detecting target polynucleotides in a sample.

### BACKGROUND

Rolling Circle Amplification (RCA) is a technique for isothermal amplification of long single-stranded concatemeric replication products from circular polynucleotide (e.g., DNA or RNA) templates. For in vitro diagnostics, the circular templates are formed by annealing single-stranded linear 5′-phosphorylated padlock probe polynucleotides head-to-tail to a linear target DNA or RNA sequence, followed by ligase-mediated joining of the ends of the padlock probes. Correct (i.e., non-mismatched at the ligation junction) annealing is required for most DNA or RNA ligases, which enables RCA to distinguish single point mutations in the target sequence. Circularized padlock probes are amplified using a start primer that anneals to the padlock probe and initiates the replication reaction by a polymerase with high strand-displacement activity, such as Phi29 polymerase, to form single-stranded antisense copies of the padlock probe sequence. The single-stranded RCA products are usually separated as high molecular weight polynucleotides with low migration in matrices, such as agarose or polyacrylamide gels or paper, and detected by using a labeled start primer or by hybridization of labeled oligonucleotide detection probes (e.g., fluorescent, biotinylated, digoxigeninated, or radiolabeled) and by enzymatic amplification in vitro as well as in situ (e.g., in paraffin-embedded tissue slides or fixed cells). However, most of these detection methods have relatively low sensitivity (e.g., only one or a few labels is usually present in the detection probe) and there is high background due to non-specific random hybridization and replication. The detection methods are also time-consuming due to the length of time needed to anneal and wash the non-specifically bound probes from the sample. There exists a need for testing methods and kits that are simple to use and provide rapid and specific results.

### SUMMARY

The disclosure provides kits and methods for detecting a target polynucleotide sequence in a sample. In one aspect, the disclosure provides a method for detecting a target polynucleotide sequence in a sample comprising: (1) a circularization step comprising combining the target polynucleotide sequence with (a) a padlock probe polynucleotide sequence comprising a 5′ end complementary to a first section of the target polynucleotide sequence and a 3′ end complementary to a second section of the target polynucleotide sequence adjacent to the first section, wherein the target polynucleotide sequence lacks a missing base selected from adenine, cytosine, guanine, thymine, uracil, and combinations thereof, and wherein the padlock probe polynucleotide sequence lacks a base complementary to said missing base, and (b) a ligase, to form a circular padlock probe; (2) an amplification step comprising combining the circular padlock probe with (a) a polymerase and (b) a mixture of deoxynucleotide triphosphates (dNTPs), wherein the mixture of dNTPs lacks said missing base, and optionally (c) a start primer comprising a polynucleotide sequence complementary to a portion of the padlock probe polynucleotide sequence, to form antisense copies of the padlock probe; and (3) a detection step comprising identifying the antisense copies of the padlock probe.

In another aspect, the disclosure provides a kit for detection of a target polynucleotide sequence in a sample comprising: (1) a padlock probe polynucleotide sequence comprising a 5′ end complementary to a first section of the target polynucleotide sequence and a 3′ end complementary to a second section of the target polynucleotide sequence located adjacent to the first section, wherein the target polynucleotide sequence lacks a missing base selected from adenine, cytosine, guanine, thymine, uracil, and combinations thereof, and wherein the padlock probe polynucleotide sequence lacks a base complementary to said missing base; (2) a ligase that anneals the 5′ and 3′ ends of the padlock probe polynucleotide together to form a circular padlock probe; (3) a polymerase; (4) a mixture of deoxynucleotide triphosphates (dNTPs) wherein the mixture of dNTPs lacks said missing base; and optionally (5) a start primer comprising a polynucleotide sequence complementary to a portion of the padlock probe polynucleotide sequence.

In one aspect, the missing base is adenine, and the mixture of dNTPs lacks deoxyadenosine triphosphate (dATP). In another aspect, the missing base is cytosine, and the mixture of dNTPs lacks deoxycytidine triphosphate (dCTP). In still another aspect, the missing base is guanine, and the mixture of dNTPs lacks deoxyguanosine triphosphate (dGTP). In one aspect the missing base is thymine, and the mixture of dNTPs lacks deoxythymidine triphosphate (dTTP). In another aspect, the missing base is uracil, and the mixture of dNTPs lacks deoxyuridine triphosphate (dUTP) or deoxythymidine triphosphate (dTTP).

In various aspects, the target polynucleotide sequence comprises DNA or RNA from a bacterial, viral, or protozoan pathogen, for example, a flavivirus (e.g., Zika virus), human papillomavirus, Chlamydia tracomatis, or Neisseria gonorrhoeae. In one aspect, the ligase is T4 DNA ligase, T4 RNA ligase 2 (Rn12), or PBCV-1 ligase. In another aspect, the polymerase is Phi29 (129) polymerase, Bst DNA polymerase, or IsoPol™ DNA polymerase (or other polymerase with high strand-displacement activity). In one aspect, the mixture of dNTPs comprises labeled dNTPs, optionally selected from the group consisting of fluorescent dNTPs, biotinylated dNTPs, digoxigeninated dNTPs, radiolabeled dNTPs, ethynyl-dNTP (e.g., 5-ethynyl-dUTP), bromo-dUTP (BrdUTP), and combinations thereof.

The foregoing summary is not intended to define every aspect of the invention, and other features and advantages of the present disclosure will become apparent from the following detailed description, including the drawings. The present disclosure is intended to be related as a unified document, and it should be understood that all combinations of features described herein are contemplated, even if the combination of features are not found together in the same sentence, paragraph, or section of this disclosure. In addition, the disclosure includes, as an additional aspect, all embodiments of the invention narrower in scope in any way than the variations specifically mentioned above. With respect to aspects of the disclosure described or claimed with “a” or “an,” it should be understood that these terms mean “one or more” unless context unambiguously requires a more restricted meaning. With respect to elements described as one or more within a set, it should be understood that all combinations within the set are contemplated. If aspects of the disclosure are described as “comprising” a feature, embodiments also are contemplated “consisting of” or “consisting essentially of” the feature. Additional features and variations of the disclosure will be apparent to those skilled in the art from the entirety of this application, and all such features are intended as aspects of the disclosure.

### BRIEF DESCRIPTION OF THE FIGURE

FIG. 1 depicts a schematic of a method of the disclosure. In a sample comprising a DNA or RNA target sequence containing only three bases (a cytosine-free target is shown), there is circularization by ligase of a linear padlock probe that contains only the three bases complementary to the bases in the target (a guanine-free padlock probe is shown). Amplification of the circularized guanine-free padlock probe sequence occurs in the presence of the other three nucleotides (dATP, dGTP, and dTTP) and labeled dNTPs (fluorescein-12-dUTP). Because there is no dCTP in the amplification reaction, amplification cannot proceed when guanine is in the template, thus reducing background from incorrectly ligated guanine-free padlock probes or from genomic DNA and RNA comprising all four bases in the sample.

FIG. 2 depicts a schematic of a two-step trinucleotide rolling circle amplification in which fragmented reaction products from a first reaction serve as (self-priming) targets for a second TN-RCA, e.g., with a circular Padlock probe.

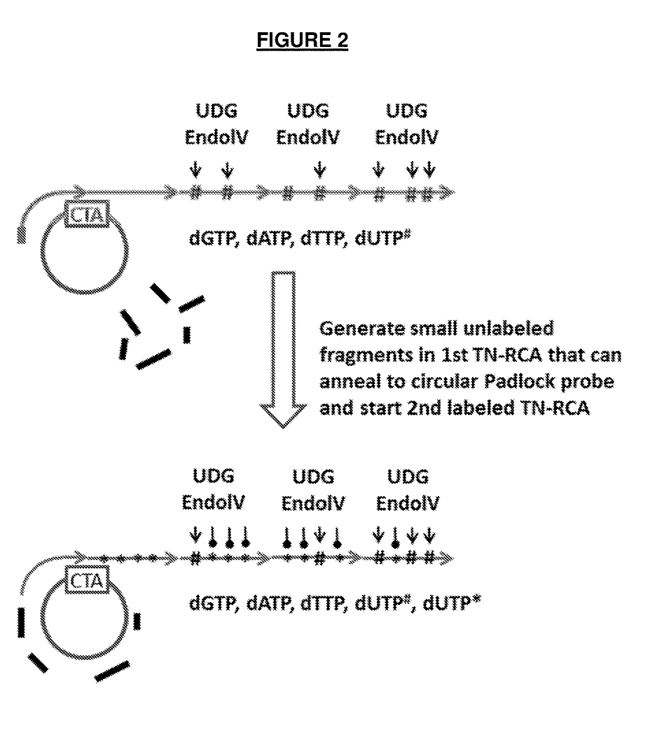

### DETAILED DESCRIPTION

The disclosure provides methods and kits for detecting a target polynucleotide sequence lacking a missing base selected from adenine, cytosine, guanine, thymine, uracil, and combinations thereof, that can be used for, e.g., rapid and sensitive sequence-specific detection, quantification and diagnosis of natural and synthetic DNA or RNA in a sample with low background. Possible applications of TN-RCA include, but are not limited to, diagnosis and detection of pathogens (bacteria, virus, parasites) in situ or in vitro (e.g., in test tubes/microfluidics), labeling and detection of proteins (e.g., antibodies), and identification and modification of objects and surfaces.

Naturally occurring DNA and RNA are polynucleotide sequences made from four types of bases: adenine, guanine, thymine, and cytosine for DNA, and adenine, guanine, cytosine, and uracil for RNA. Complementary base pairs are formed from specific bonding between adenine and thymine/uracil and between cytosine and guanine in sense and antisense polynucleotide sequences. Long (e.g., more than 20 bases or base pairs in length) stretches of polynucleotide sequences comprising only three of the four types of bases (i.e., tri-nucleotide) are rare. In the methods and kits of the present disclosure, target polynucleotide sequences lacking a missing base selected from adenine, cytosine, guanine, thymine, uracil, and combinations thereof are sequence-specifically recognized, transferred, extended, amplified and detected using padlock probe polynucleotide sequences that consist entirely of bases complementary to the nucleotides present in the target sequence.

The 5′-phosphorylated padlock probes anneal sequence-specifically head-to-tail with their ends to the target polynucleotide sequence and completely lack the base complementary to the missing base. Upon specific head-to-tail annealing and ligation to the target polynucleotide, the padlock probes provided extended complementary circular versions of the target polynucleotide that can be further amplified by RCA into long, linear single stranded polynucleotide products comprising repetitive, antisense copies of the padlock probe polynucleotide sequence. The missing base, in its deoxynucleotide triphosphate (dNTP) form, is not added to the reaction mixture during polymerization, so only the correctly ligated circular padlock probe polynucleotide sequence is amplified, and no background amplification occurs with endogenous RNA or DNA (which would require the presence of all four dNTPs) present in the sample.

The methods of the disclosure thus provide increased specificity of amplification and lower background compared to conventional RCA. In RCA, background signals are generated from the presence of high molecular weight genomic DNA, which co-migrate with the RCA product and can result from amplification due to the presence of nicks in genomic DNA or from non-specific annealing and/or amplification of padlock probe, start primer, or detection probe to genomic DNA or RNA. Due to the presence of only three or fewer dNTPs in the amplification reaction of the methods disclosed herein, only correctly ligated circular padlock probes will amplify and incorporate labeled dNTPs into the amplification product, which increases the specific signal and lowers the signals from background amplification from endogenous RNA/DNA. Moreover, the dNTPs and polymerase molecules are not consumed in non-specific incorporations, thereby promoting specific amplification. In general, lower concentrations of dNTPs and labeled dNTPs can be used with more favorable ratios between labeled dNTPs over unlabeled dNTPs, which increases the sensitivity of the assay and lowers the cost. Thus, in the methods of the present disclosure, a specifically labeled amplification product can be separated, detected and quantified in the presence of various other background signals, which as commonly found in, for example, biological samples.

The methods of the present disclosure also provide increased sensitivity, higher speed and lower cost compared to conventional RCA. Labeled dNTPs have been used in traditional RCA for detection, but in complex samples, they may be incorporated non-specifically and give high background. In the methods of the present disclosure, this does not occur; labeled dNTPs can be added to the RCA reaction to facilitate the separation, isolation and detection of the amplified sequences. When compared to using labeled detection probes in conventional RCA that usually contain one label per probe and, thus, also one label per detected amplification product, the methods of the present disclosure can incorporate many and multiple-type labeled dNTPs. Since the label is incorporated during the amplification step, less time is required for detection because no hybridization is required, and no specific detection probe needs to be synthesized, thereby lowering the assay cost.

Another advantage of the methods of the present invention is increased specificity at lower reaction temperatures. For example, guanine and cytosine form triple-bonds with high affinity in DNA and RNA, and when either is the missing base, the overall melting temperature is lower, with consequent lower secondary structure of the padlock probe and target sequences, as well as lower self-priming and self-annealing features of the padlock probe. Similarly, the start primer, if used, has a lower secondary structure and self-dimer and self-priming features, again reducing background. These features allow for annealing of the ends of the padlock probes at lower temperature (e.g., 20° C. to 40° C.) and/or the usage of longer padlock probe ends for annealing to the target sequences, which leads to higher specificity and sensitivity. The absence of one base in the padlock probe and start primer also reduces their mis-alignment to non-target DNA and RNA in biological samples that mostly consist of sequences containing all four bases, thus increasing sensitivity by reducing background from mis-priming.

The methods and kits of the disclosure are also suitable for point-of-care tests, for example, as clinical, laboratory or field kits, or as research and analytical techniques for in vitro and in situ measurement of specific DNA or RNA. The methods and kits of the disclosure can be used for in situ detection, quantification and localization of target DNA or RNA in tissue sections (e.g., frozen and paraffin-embedded tissue sections), in fixed cells, and in dried samples (e.g., dried urine, saliva, blood or other forensic samples), as well as with microfluidics or automated microtiter-based platforms. Additionally, the methods and kits of the disclosure can be used to detect, modify, and identify/authenticate other molecules (e.g., antibodies, proteins, lipids, nucleic acids, organisms (including genetically modified organisms), chemicals, solutions such as color in paintings or biometric ink in writings, surface properties for bioengineering applications, such as beads, chips, microarrays, conductors, semi-conductors, velcro-type molecular fasteners, etc.) that have been tagged, modified or spiked with synthetic polynucleotides (e.g., stabilized with phosphothionate linkage) encoding the complementary target sequence. Similar PCR-based techniques for encryption, labeling, and coding exist, however, they require longer pieces of DNA (100-500 bp) what makes them more expensive, less stable and easier to decode by conventional sequencing compared to the methods of the disclosure.

The methods of the present disclosure are also compatible for use in accordance with the methods and kits described in International Patent Application No. PCT/US2016/20464, incorporated herein by reference.

The following definitions may be useful in aiding the skilled practitioner in understanding the disclosure. Unless otherwise defined herein, scientific and technical terms used in the present disclosure shall have the meanings that are commonly understood by those of ordinary skill in the art.

The term “polynucleotide” refers to a single-stranded or double-stranded polymeric chain of nucleotides and can comprise naturally occurring or synthetic DNA or RNA, other synthetic nucleic acids or nucleic acid analogs, or a combination of any of the foregoing.

The term “padlock probe” refers to a single-stranded polynucleotide whose 5′ and 3′ ends are complementary to a target polynucleotide sequence, for example, as described in Nilsson et al., Science 265(5181):2085-2088 (1994), incorporated herein by reference.

The term “start primer” refers to a polynucleotide that hybridizes to a reference polynucleotide sequence and serves as the starting point for synthesis of antisense copies of the reference polynucleotide sequence.

The term “rolling circle amplification” or “RCA” refers to the isothermal amplification of a circularized probe, for example, as described in U.S. Pat. Nos. 5,854,033 and 6,783,943, incorporated herein by reference.

In one aspect, the disclosure provides a method for detecting a target polynucleotide sequence in a sample comprising: (1) a circularization step comprising combining the target polynucleotide sequence with (a) a padlock probe polynucleotide sequence comprising a 5′ end complementary to a first section of the target polynucleotide sequence and a 3′ end complementary to a second section of the target polynucleotide sequence adjacent to the first section, wherein the target polynucleotide sequence lacks a missing base selected from adenine, cytosine, guanine, thymine, uracil, and combinations thereof, and wherein the padlock probe polynucleotide sequence lacks a base complementary to said missing base; and (b) a ligase, to form a circular padlock probe; (2) an amplification step comprising combining the circular padlock probe with (a) a polymerase, and (b) a mixture of deoxynucleotide triphosphates (dNTPs), wherein the mixture of dNTPs lacks said missing base; and optionally (c) a start primer comprising a polynucleotide sequence complementary to a portion of the padlock probe polynucleotide sequence, to form antisense copies of the padlock probe; and (3) a detection step comprising identifying the antisense copies of the padlock probe.

In another aspect, the disclosure provides a kit for detection of a target polynucleotide sequence, which is optionally a pathogen polynucleotide sequence, in a sample comprising: (1) a padlock probe polynucleotide sequence comprising a 5′ end complementary to a first section of the target polynucleotide sequence and a 3′ end complementary to a second section of the target polynucleotide sequence located adjacent to the first section, wherein the target polynucleotide sequence lacks a missing base selected from adenine, cytosine, guanine, thymine, uracil, and combinations thereof, and wherein the padlock probe polynucleotide sequence lacks a base complementary to said missing base; (2) a ligase that anneals the 5′ and 3′ ends of the padlock probe polynucleotide together to form a circular padlock probe; (3) a polymerase; (4) a mixture of deoxynucleotide triphosphates (dNTPs), wherein the mixture of dNTPs lacks said missing base; and optionally (5) a start primer comprising a polynucleotide sequence complementary to a portion of the padlock probe polynucleotide sequence. The (4) mixture of dNTPs may include modified bases such as inosine or 2′-Deoxyuridine-5′-Triphosphate (dUTP). Modified bases also may be used in the Padlock sequence, in particular in the end-sequences annealing to the target. For example, “Universal base analogues” (Nucleic Acids Research, 2001, 29, 2437-2447) allow annealing to a target having mismatches with increased stability. In particular, the incorporation of “Universal base analogues” into the end-sequences of the Padlock sequence can be used when the trinucleotide-target sequence contains one or a few of the fourth, otherwise missing, base. In this situation, the “Universal base analogues” opposite the occasional fourth base present in the trinucleotide target sequence will facilitate annealing, but still only three dNTP are required for TN-RCA amplification, thus increasing the number of potential target sequences that are accessible to TN-RCA.

In various aspects, the target polynucleotide sequence comprises DNA and/or RNA, including one or a combination of naturally occurring polynucleotides and fragments thereof, synthetic polynucleotides, single-stranded polynucleotides, or double-stranded polynucleotides. In one aspect, the target polynucleotide sequence is from any bacterial, fungal, or viral pathogen. For example, in one aspect, the methods and kits of the disclosure are used to detect a bacterial pathogen including, but not limited to, bacteria belonging to the genus Bacillus (e.g., B. anthracis), Bordetella (e.g. B. pertussis), Borrelia (e.g., B. burgdorferi), Brucella (e.g., B. abortus, B. canis, B. melitensis, B. suis), Campylobacter (e.g., C. jejuni), Chlamydia (e.g., C. pneumonia, C. psittaci, C. trachomatis), Clostridium (e.g., C. botulinum, C. difficile, C. perfringens), Corynebacterium (e.g., C. diphtheria), Enterococcus (e.g., E. faecalis), Escherichia (e.g., E. coli), Gardnerella (e.g., G. vaginalis), Haemophilus, (e.g., H. influenza), Helicobacter (e.g., H. pylori), Legionella (e.g., L. pneumophila), Listeria, (e.g., L. monocytogenes), Mycoplasma (e.g., M. genitalium), Neisseria (e.g., N. gonorrhoeae, N. meningitides), Pseudomonas (e.g., P. aeruginosa), Salmonella (e.g., S. typhi, S. typhimurium), Staphylococcus (e.g., S. aureus), Streptococcus (e.g., S. pneumonia), or Vibrio (e.g., V. cholerae). In another aspect, the methods and kits of the disclosure are used to detect a fungal pathogen including, but not limited to, fungi belonging to the genus Aspergillus, Blastomyces, Candida, Cladosporium, Coccidioides, Cryptococcus, Exserohilum, Histoplasma, Mucoromycotina, Pneumocystis, Sporothrix, or Stachybotrys. In still another aspect, the methods and kits of the disclosure are used to detect a viral pathogen including, but not limited to, adeno-associated virus, Ebolavirus, encephalomyocarditis virus, Epstein-Barr virus, hepatitis virus, herpesvirus (e.g., herpes simplex virus Type 1 or Type 2), human immunodeficiency virus (HIV), human papillomavirus (HPV), influenza virus, MERS coronavirus, measles virus, mumps virus, Norovirus, poliovirus, rotavirus, rubella virus, or a flavivirus (e.g., Zika virus, West Nile virus, yellow fever virus, dengue virus, encephalitis virus); or a protozoan pathogen including, but not limited to, Trichomonas vaginalis. In another aspect, the methods and kits of the disclosure are used to detect a plant and/or a pathogen of plants, including, but not limited to, plants of agricultural importance and pathogens thereof.

The genomes of the representative pathogens referenced above are known in the art, and identification of a target polynucleotide sequence lacking a missing base selected from adenine, cytosine, guanine, thymine, uracil, and combinations thereof, and a corresponding padlock probe with complementary sequences suitable for hybridization to the target polynucleotide sequence, is well within the skill of the art. In one aspect, the pathogen polynucleotide comprises a target polynucleotide selected from the genome of Zika virus (e.g., (Genbank NC_012532.1, KJ776791.1, or KU497555.1), HPV (e.g., GenBank K02718.1), Chlamydia tracomatis (e.g., GenBank CP015304.1), or Neisseria gonorrhoeae (e.g., GenBank CP016015.1), such as the polynucleotide of SEQ ID NO: 1, 2, 3, or 10.

In one aspect, the target polynucleotide sequence is about 20 to about 40 bases in length, for example, about 20 to about 30, about 25 to about 40, about 30 to about 40, about 25 to about 35, or about 20 to about 35 bases in length. In another aspect, the padlock polynucleotide sequence is about 50 to about 200 bases in length, for example, about 50 to about 100, about 75 to about 150, about 50 to about 150, about 100 to about 200, or about 75 to about 200 bases in length. For example, in various aspects, the target polynucleotide is at least 20 bases, at least 25 bases, at least 30 bases, at least 35 bases, at least 45 bases, or at least 50 bases in length, and is no more than 200 bases, no more than 175 bases, no more than 150 bases, no more than 125 bases, no more than 100 bases, no more than 75 bases, no more than 50 bases, or no more than 40 bases in length.

In one aspect, the target polynucleotide sequence is present in or obtained from a biological sample. The biological sample is, in various embodiments, obtained from a human or other mammalian subject, or from any other organism including plants, e.g. carrying a pathogen (e.g. virus- or bacteria-infected insects or plants) for example, by collecting a fluid or tissue sample (e.g., collecting a blood, urine, amniotic fluid, or saliva sample) or swabbing a body orifice. The sample may be collected by a health care worker or researcher, or by self-sampling, e.g., by patients or agricultural workers. Alternatively, the biological sample is obtained from an environmental source, such as water or soil or a plant. The biological sample may also be a food sample (e.g., a fluid or swab taken from food in order to detect contamination).

In an optional denaturation/renaturation step prior to the circularization step, the target polynucleotide sequence is denatured (e.g., by heat or alkali for about 5 to about 10 minutes) and then renatured by neutralization buffer. Alternatively, the single-stranded target polynucleotide sequence is generated by digesting the strand complementary to the target sequence by using nicking enzymes and Exonuclease III, e.g., as described in Christian et al., PNAS 98: 14238-14243 (2001), incorporated herein by reference. The requirements of this step depend on whether the target polynucleotide sequence is single-stranded or double-stranded DNA or RNA or has secondary structure. In one aspect, high molecular weight polynucleotides (e.g., genomic DNA) are physically (e.g., by acoustic shearing, ultrasonication, or hydrodynamic shear) or enzymatically (e.g., by non-specific nuclease(s) and/or T7 endonuclease) fragmented and/or cut by specific restriction endonucleases or nicking endonucleases, optionally at sites close to the target sequence, before denaturation. In another aspect, RNA is fragmented chemically by heat and divalent metal cations (e.g., magnesium or zinc), or enzymatically by RNase III digestion, or RNA is converted to cDNA before denaturation and RNase digestion. In another aspect, whole-genome amplification is used to generate single stranded target DNA. Double-stranded genomic DNA can also be opened and made accessible to Padlock annealing with peptide nucleic acid (PNA) probes.

In the circularization step, the target polynucleotide sequence that lacks a missing base selected from adenine, cytosine, guanine, thymine, uracil, and combinations thereof, is combined with a padlock probe polynucleotide sequence comprising a 5′ end complementary to a first section of the target polynucleotide sequence and a 3′ end complementary to a second section of the target polynucleotide sequence adjacent to the first section, wherein the padlock probe polynucleotide sequence lacks a base complementary to said missing base, and a ligase. In one aspect, the target polynucleotide sequence lacks adenine, and the padlock probe polynucleotide sequence lacks thymine. In another aspect, the target polynucleotide sequence lacks adenine, and the padlock probe polynucleotide sequence lacks uracil and thymine. In still another aspect, the target polynucleotide sequence lacks thymine, and the padlock probe polynucleotide sequence lacks adenine. In another aspect, the target polynucleotide sequence lacks uracil, and the padlock probe polynucleotide sequence lacks adenine. In one aspect, the target polynucleotide sequence lacks cytosine, and the padlock probe polynucleotide sequence lacks guanine. In another aspect, the target polynucleotide sequence lacks guanine, and the padlock probe polynucleotide sequence lacks cytosine.

The 5′ end and 3′ end of the padlock probe hybridize to adjacent first and second sections of the pathogen polynucleotide sequence. In various aspects, the padlock probe comprises a polynucleotide sequence selected from the group consisting of SEQ ID NOs: 4 and 11. In one aspect, the padlock probe comprises a 5′ section comprising the sequence in SEQ ID NO: 5 or 12 and/or a 3′ section comprising the sequence set forth in SEQ ID NO: 6 or 13. In another aspect, the padlock probe comprises the polynucleotide sequences of SEQ ID NOs:5 and 6. In still another aspect, the padlock probe comprises the polynucleotide sequences of SEQ ID NOs: 12 and 13. Variants comprising a nucleic acid sequence comprising at least about 80%, at least about 90%, or at least about 95% sequence identity to the sequences referenced above also may be used in the context of the invention. Because the padlock probe lacks at least one base type, there is no self-annealing and self-ligation of the padlock probe, which can be a problem with conventional RCA.

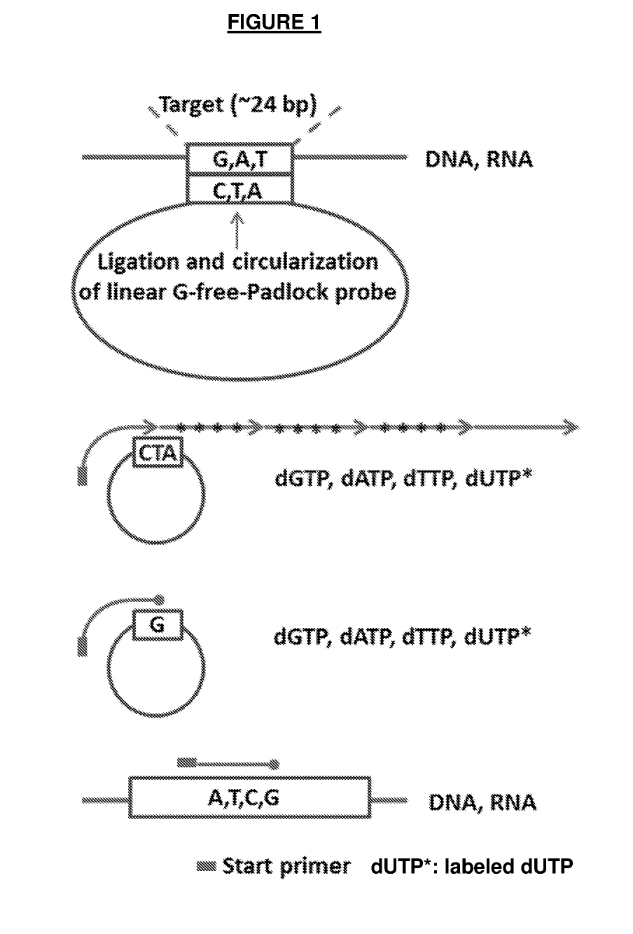

The hybridization of the padlock probe to the target polynucleotide brings the 5′ end and 3′ end of the padlock probe in close proximity, allowing the ligase to join the 5′ end and 3′ end of the padlock probe together to form a circular padlock probe (FIG. 1). The target polynucleotide sequence is only briefly required to serve as a bridge to circularize the padlock probe for subsequent amplification. In the case of self-priming, the target polynucleotide sequence serves as a potential starting point of amplification. It will be appreciated that the padlock probe polynucleotide sequences described herein are merely examples of sequences suitable for use in a padlock probe, and other sequences are suitable for use so long as the sequences hybridize to the target polynucleotide sequence in such a manner as to allow a ligase to generate a circular padlock probe. In one aspect, the ligase is an enzyme that can ligate polynucleotide strands at a temperature at or below 40 ° C., for example T4 DNA ligase, T4 RNA ligase 2 (Rn11), or PBCV-1 (Chlorella virus) ligase. In another aspect, circular padlock probes are generated using CIRCLIGASE ssDNA Ligase (Epicentre, Madison Wis.).

In one aspect, the target polynucleotide sequence is genomic DNA, and the DNA is cleaved with a restriction endonuclease or nicking endonuclease close to the 3′-side of the padlock probe and then denatured and annealed to the target as outlined above. Annealing of DNA/DNA generates B-DNA type helix of 10 bp per helical turn, which can be ligated efficiently by T4 ligase and 10 mM ATP in the presence of fresh DTT. During amplification by the polymerase, cutting the target DNA polynucleotide sequence close to the 3′ side of the padlock probe polynucleotide sequence facilitates the release of the circular padlock probe from the intertwined genomic DNA and generates a 3′-end for self-priming. For DNA targets, to ensure a 3′-end close to the target sequence, ssDNA can be cleaved by DNA Glycosylase and Endonuclease IV, e.g., as described in Andersson et al., Virology 426:87-92 (2012), incorporated herein by reference. The generated 3′-end of the target sequence can serve as the starting point of the amplification reaction in the absence of the start primer, what can reduce background coming from short extensions by start primer and unligated padlock probe. Cutting of target DNA can also be achieved by annealing Padlock probes with a T/A mismatch and subsequent cleavage of the Adenine in the target sequence by MutY adenine DNA glycosylase. Whole genome amplification and protein nucleic acid (PNA) probes can be used to make the target more accessible for the Padlock probes.

In various aspects of the method, the mixture of dNTPs in the amplification step comprises dUTP, and the method further comprises, prior to the detection step, cleaving the product of the initial amplification step with Uracil-DNA-glycosylase to generate single-stranded amplification products. These single-stranded amplification products can serve as self-priming targets for a second TN-RCA reaction, optionally with preligated circular Padlock probes. The amplification step is then repeated on the single-stranded amplification products.

In another aspect, the target polynucleotide sequence comprises RNA, the padlock polynucleotide sequence comprises DNA, and PBCV-1 Ligase from Chlorella virus is used. Alternatively, T4 ligase with low ATP (e.g.,10 μM) and NaCl with high MgCl₂ (e.g., 10 mM) and fresh DTT is used. SplintR® Ligase also may be used. Annealing of DNA/RNA generates A-DNA-like structure having intermediate characteristics between the A- and B-DNA-type structure and between 10 and 11.6 bp per helical turn which is preferentially ligated by PBCV-1 Ligase. An RNase such as RNase H, RNase A, and/or RNase III is then added, which specifically hydrolyzes the RNA in RNA/DNA hybrids, thus releasing the circular padlock probe form the intertwined RNA. When T4 ligase is used with an RNA target, the ATP concentration is optionally reduced to 10 microM to minimize inhibitory AppLigase complexes. Moreover, the intrinsic 3′ to 5′ RNase activity of Phi29 polymerase can be used to digest the RNA that is not hybridized to the target sequence, generating 3′-ends that can be used as starting point for synthesis of antisense copies of the reference polynucleotide sequence in the absence of start primer, e.g., as described in Lagunavicius et al., RNA 14: 503-513 (2008), incorporated herein by reference. Accordingly, with DNA, self-priming can be initiated after digesting the non-annealed DNA by a 3′ to 5′ exonuclease, such as Phi29.

In the amplification step, the circular padlock probe is combined with a polymerase with high strand-displacement activity and a mixture of dNTPs lacking the base missing from the target polynucleotide sequence, to form a single-stranded polynucleotide sequence containing repetitive, antisense copies of the padlock probe polynucleotide sequence. Optionally, a start primer comprising a polynucleotide sequence complementary to a portion of the padlock probe polynucleotide sequence is added during the amplification step to initiate replication. In various aspects, the start primer comprises a polynucleotide sequence set forth in SEQ ID NO: 7, or a polynucleotide sequence complementary to a portion of SEQ ID NO: 4 or 11. Primers comprising a sequence at least 90% identical to these sequences also may be used in various embodiments.

In another aspect, no start primer is used, which serves to reduce background from amplification of non-ligated padlock probes annealed to start primer. For example, for short RNA/DNA targets, the 3′-end of the target oligonucleotide can serve as efficient starting point for self-priming so that no start primer is required. Moreover, since Phi29 polymerase has 3′ to 5′ RNase activity which digests ssRNA, the non-digested RNA remaining annealed to the ligated Padlock probe can serve as start site of the TN-RCA reaction; consequently, even for longer target RNA, a start primer is not required and can be absent in the reaction mixture. For longer RNA targets, digestion can be facilitated by adding specific RNases (e.g., RNase III, RNase H), whereas longer DNA targets may require fragmentation by restriction endonucleases, nickases, or dsDNA Fragmentase (NEB). For DNA targets, a self-priming 3′-end can also be generated by annealing Padlock probes with a T/A mismatch and subsequent cleavage of the Adenine in the target sequence by MutY adenine DNA glycosylase.

In one aspect, the target polynucleotide sequence lacks adenine, the padlock probe polynucleotide sequence lacks thymine, and the mixture of dNTPs lacks adenine in the form of deoxyadenosine triphosphate (dATP). In another aspect, the target polynucleotide sequence lacks adenine, the padlock probe polynucleotide sequence lacks uracil, and the mixture of dNTPs lacks dATP. In still another aspect, the target polynucleotide sequence lacks thymine, the padlock probe polynucleotide sequence lacks adenine, and the mixture of dNTPs lacks thymine in the form of deoxythymidine triphosphate (dTTP) and uracil in the form of deoxyuridine triphosphate (dUTP). In another aspect, the target polynucleotide sequence lacks uracil, the padlock probe polynucleotide sequence lacks adenine, and the mixture of dNTPs lacks uracil in the form of deoxyuridine triphosphate (dUTP). In one aspect, the target polynucleotide sequence lacks cytosine, the padlock probe polynucleotide sequence lacks guanine, and the mixture of dNTPs lacks cytosine in the form of deoxycytidine triphosphate (dCTP). In another aspect, the target polynucleotide sequence lacks guanine, the padlock probe polynucleotide sequence lacks cytosine, and the mixture of dNTPs lacks guanine in the form of deoxyguanosine triphosphate (dGTP).

With a base missing in the mixture of dNTPs, the polymerase will only form long single stranded extension products from circularized padlock probes because replication of other sequences will terminate when the missing base is required for complementary base pairing, which reduces background signals, e.g., derived from nicked 3′ ends present in endogenous RNA or DNA in the sample or from mis-priming by the start primer. Since there is no background amplification and the RCA can only proceed with the correctly ligated circular padlock probe, labeled dNTPs can be added to generate labeled high molecular weight single stranded RCA products for sensitive and specific detection. Examples of labeled dNTPs include, but are not limited to fluorescent dNTPs (e.g., Fluorescein-12-dNTPs), biotinylated dNTPs (e.g., Biotin-11-dNTPs), digoxigeninated dNTPs, radiolabeled dNTPs, ethynyl-dNTP (e.g., 5-ethynyl-dUTP), bromo-dUTP (BrdUTP), and combinations thereof. The amount of incorporation of the labeled nucleotides and, thus, the sensitivity of the assay can be improved by increasing the number of complementary nucleotide templates in the padlock probe polynucleotide sequence, the concentrations of the labeled dNTPs, or the time of the amplification reaction. The replication of single stranded antisense copies of the padlock probe is mediated by the polymerase. In one aspect, the polymerase is capable of working on small circular polynucleotides and has high strand displacement activity, for example, a polymerase derived from a bacteriophage or bacterium, such as Phi29 (129) polymerase or Bacillus stearothermophilus (Bst) DNA polymerase (e.g., Bst DNA polymerase, large fragment) or mutants of the aforementioned polymerase retaining polymerase activity.

In an optional separation step following the amplification step, the single stranded antisense copies of the padlock probe are separated from the reaction mixture by any separation method, including, but not limited to, agarose or polyacrylamide gels, paper strips, DNA-affinity columns, nucleotide exclusion columns, streptavidin-coated beads (e.g., magnetic or styropor beads) or microtiter plates, or ethanol precipitation.

In the detection step, the single-stranded antisense copies of the padlock probe are identified. In one aspect, labeled single stranded amplification products are optionally separated and detected by UV illumination. For example, dNTPs can be labeled with fluorescein, which has absorbance and emission wavelengths of 495 nm and 520 nm, respectively, and can be detected using a green channel filter. Alternatively or in addition, biotinylated or digoxigeninated single stranded amplification products can be detected by enzyme-coupled streptavidin (e.g., horseradish peroxidase (HRP)-coupled streptavidin or alkaline phosphatase-coupled streptavidin or using enzyme-coupled anti-digoxigenin, anti-fluorescein, or anti-biotin antibodies), which form visible reaction products. The uncoupled versions of these streptavidins and antibodies alone and in combination can also be used to catch, concentrate and immobilize the labeled amplification products at specific surfaces and locations for subsequent detection by enzyme- or fluorescein-coupled streptavidins or antibodies. The HRP reaction can also be visualized by exposure to conventional or Polaroid film or measured by electrochemical detection. The binding of labeled amplification products to surfaces can also be detected by altering the surface properties, such as electrical conductance, light diffraction and reflection, aggregation, color, pH change, etc.

In one example, an agarose gel may be used. High molecular weight TN-RCA reaction products are efficiently separated from all the background signals by 1.5% agarose gels, and when labeled Fluorescein-12-dUTP is added to the reaction, no Ethidium Bromide or Gel Red is necessary to visualize the products in the gel. In another example, biotinylated TN-RCA reaction products are added to the well of a microtiter plate and incubated, e.g., for 20 minutes. The well is washed 3 times with 200 μL PBST (1 tablet PBS (Invitrogen) in 100 ml H2O and 100 μL (0.1%) of Tween-20, filtered with a 0.2 μm filter) and blocked with 200 μL PBST/1% BSA for 10 min. Detection can be done with the Opti-4CN™ substrate kit (Biorad) by adding 150 μL of 1/1000 dilution of Blotting grade Avidin-HPR in Antibody dilution buffer (PBST/1% BSA) for 20 min, washed with 200 μL PBST for 3×5 min, and detected by adding 0.2 ml of Opti-4CN substrate per 10 ml diluents (mixed one part of Opti-4CN diluent with 9 parts H2O). The plate is incubated for up to 30 min with shaking. Alternatively, instead of the microtiter plate, the sample can be dot blotted onto Nitrocellulose membranes and air dried and detected as above using the Opti.4CN kit according to the manufacturers' protocol (Biorad). This method gives no background when the ligase is omitted in the TN-RCA reaction, but it gives a very low background without target DNA/RNA and TN-RCA. This background is most likely due to the extension of the Padlock probe with the Start primer, which leads to incorporation of some Biotin-11-dUTP up to the end of the unligated Padlock probe. In addition, with (synthetic) short target RNA/DNA, signals may be generated by annealing the target to Padlock probe and extension to the end of the Padlock probe. Background can be avoided by leaving away the Start primer and use instead self-priming, e.g. after digestion of the target with various RNases, that digest the non-annealed target generating a free 3′-end for priming. In another example, separation can be performed using a DNA affinity column based on the Monarch DNA PCR and DNA cleanup kit (Monarch, NEB), with detection based on the Opti-4CN detection kit (Biorad). For example, 60 microL of the 11-Biotin-dUTP labeled TN-RCA reaction is added to 420 microL of binding buffer, then loaded on spin column and centrifuged for 1 min. The column is washed 2× with 200 microL wash buffer and spun for 1 min in between. Then, 200 microL of 1/1000 dilution of Blotting grade Avidin-HPR in Antibody dilution buffer (PBST/1% BSA) from the Opti-4CNTM Substrate Kit (Biorad) is added, centrifuged for one minute, washed 2× with 200 microL binding buffer (Monarch, NEB) (spin 1 min in between). Then, one part of Opti-4CN diluent with 9 parts H2O is prepared and 0.2 ml of Opti-4CN substrate per 10 ml of diluents is mixed, 200 microL added to the spin column and incubated for 5-30 min with and the spin column dried with paper (spinning here will wash out the colored dye as well, but it can be restained again). As alternative to the centrifugation step that requires a centrifuge and electricity, the filter is attached to a 10 ml syringe, and the same procedure is performed by manually generating a vacuum and sucking all the solutions into the syringe.

In one embodiment, a labeled (e.g., fluorescein, biotin, digoxigenin, gold-nanoparticle) reporter or detection probe (e.g., Biotin-5-CTCAACCTTACTACACTC-3 (SEQ ID NO: 20)) is added to the running buffer, and then hybridizes to the antisense copies of the padlock probe to form a reporter complex. In one aspect, the reporter probe comprises a polynucleotide sequence identical to a region of the padlock probe, such as the region adjacent to the 5′ end of the padlock probe that is complementary to the pathogen polynucleotide. Optionally, the reporter probe comprises a polynucleotide sequence identical to a region of at least 10 polynucleotides of the padlock probe, for example, at least 10 polynucleotides, at least 15 polynucleotides, at least 20 polynucleotides , at least 25 polynucleotides, or at least 30 polynucleotides. Other suitable reporter probes may be generated using routine laboratory methods. Optionally, the reporter probe is conjugated to a microparticle. In one aspect, the microparticle has a diameter less than about one micrometer. In various aspects, the microparticle is selected from a nylon microparticle, a dendrimer microparticle, a styropor microparticle, a gold microparticle, a cellulose microparticle, or a magnetic (e.g., paramagnetic) microparticle. Combinations of microparticles may also be used. Conjugation of the reporter probe and microparticle can be achieved using any suitable method, such as covalent linkage. In one aspect, the detection step comprising wicking a mixture comprising reporter complexes, e.g., via capillary action, into a test strip and visually detecting the reporter probe. In one aspect, the test strip is a paper strip, optionally comprising filter paper, such as Whatman #1 filter paper. The test strip optionally comprises pores having a diameter of about 5 micrometers to about 20 micrometers, for example, about 5 micrometers, about 10 micrometers, about 11 micrometers, about 12 micrometers, about 13 micrometers, about 14 micrometers, about 15 micrometers, or about 20 micrometers. Optionally, the test strip comprises a region comprising chitosan, which non-specifically binds polynucleotides and provides a control region or indicator of test completion. The test strip separates the components in the mixture based on size exclusion so that reporter probes hybridized to amplified polynucleotides, i.e., the reporter complexes, travel less along the length of the test strip than smaller, uncomplexed reporter probes. One example of a test strip is the Milenia Hybridetect Dipstick.

In one aspect, the circularization step, amplification step, or both are performed at a temperature less than about 65° C., for example, less than about 60° C., less than about 55° C., less than about 50° C., less than about 45° C., less than about 40° C., less than about 35° C., less than about 30° C., or less than about 25° C. In some aspects, the circularization step, amplification step, or both are performed at a temperature between about 20° C. to about 40° C., about 22° C. to about 37° C., about 30° C. to about 40° C., about 20° C. to about 30° C., about 22° C. to about 35° C., about 23° C. to about 32° C., or about 25° C. and about 30° C. Traditional PCR assays require laboratory equipment to achieve temperatures greater than 90° C. The circularization step and amplification step of the present methods can, therefore, be performed with less laboratory equipment and outside clinical settings. In some aspects, the overall assay time for the methods of the present disclosure is within about 30 minutes to about 4 hours, for example, within about 1 hour, about 1.5 hours, about 2 hours, about 2.5 hours, about 3 hours, or about 3.5 hours, although in various embodiments the assay time is about 8-24 hours.

For specific applications, the methods and kits of the present disclosure comprise target polynucleotide sequences missing two or three bases selected from adenine, cytosine, guanine, thymine, and uracil, e.g., for the detection in genomic DNA or RNA of dinucleotide repeats or mononucleotides, such as polyAAA tails. In one aspect, the target polynucleotide sequence is combined with a padlock probe with complementary di-or mononucleotide sequences at the ends for annealing to the target polynucleotide sequence, but either mono-, di- or tri-nucleotide sequences in the remaining padlock probe sequence. In one aspect, target sequences missing one base are generated in DNA or RNA by chemical reactions, e.g., by deamination by sodium bisulfite of cytosine to uracil but not of 5-methylcytosine, to distinguish unmethylated and methylated target sequences.

In various aspects, the methods and kits of the disclosure comprise a variant of a polynucleotide described herein, the variant having a nucleic acid sequence comprising at least 80%, at least 85%, at least 90%, at least 95%, or at least 98% sequence identity to any of the polynucleotides set forth in SEQ ID NOs: 1-15.

Most natural DNA and RNA are devoid of long Tri-Nucleotide (TN) stretches (20-200 bp) of sequences that lack one specific nucleotide (Missing Nucleotide (MN)). There are exceptions, however, such as disease-associated trinucleotide expansions, polyAAA tails of mRNA or as present in some pseudogenes. The methods described herein are also suitable for these instances, although specific adaptations may be required. For specific applications, Di-Nucleotide RCA (DN-RCA) or even Mono-Nucleotide RCA (MN-RCA) is contemplated, e.g., for the detection in genomic DNA or RNA of dinucleotide repeats or polyAAA tails, respectively. Dinucleotide targets can be detected using Padlock probes with complementary dinucleotide sequences at the end for annealing, but trinucleotides sequences in the remaining sequence, thus allowing amplification. In particular, TN-, DN-, or MN-RCA can be used to detect synthetic oligonucleotides that have been linked to antibodies (Immuno-RCA) or to microarrays (Surface-RCA).

The following examples are provided by way of illustration and are not intended to be limiting.

### EXAMPLES

### Exemplary Detection Methods

Exemplary materials and methods for practicing various embodiments of the disclosure are provided below. Examples of detection methods include the following.

Agarose gel: High molecular weight TN-RCA reaction products are efficiently separated from all the background signals by 1.5% agarose gels, and when labeled Fluorescein-12-dUTP is added to the reaction, no Ethidium Bromide or Gel Red is necessary to visualize the products in the gel. Images of agarose gel without Ethidium Bromide or Gel Red were acquired using the Epi Blue setting by an Azure Biosystems C200 Imaging system.

Microtiter plate: 60 microL of the biotinylated TN-RCA reaction was added to the well of a Neutravidin Microtiter plate (Pierce® Neutravidin Coated High Binding Capacity (HBC) White 96-well Plates with SuperBlock® blocking buffer) and incubated for 20 minutes. The well was washed 3 times with 200 microL PBST (1 tablet PBS (Invitrogen) in 100 ml H2O and 100 microL (0.1%) of Tween-20, filtered with a 0.2 micron filter) and blocked with 200 microL PBST/1% BSA for 10 min. Detection was performed with the Opti-4CN™ substrate kit (Biorad) by adding 150 microL of 1/1000 dilution of Blotting grade Avidin-HPR in Antibody dilution buffer (PBST/1% BSA) for 20 min, washed with 200 microL PBST for 3×5 min, and detected by adding 0.2 ml of Opti-4CN™ substrate per 10 ml diluents (mixed one part of Opti-4CN diluent with 9 parts H₂O). The plate was incubated for up to 30 min with shaking and documented. Alternatively, instead of the microtiter plate, the sample can be dot blotted onto Nitrocellulose membranes and air dried and detected as above using the Opti.4CN kit according to the manufacturers' protocol (Biorad (or similar detection system)). This method gives no background when the ligase is omitted in the TN-RCA reaction, but it gives a very low background without target DNA/RNA and TN-RCA. This background is most likely due to the extension of the Padlock probe with the Start primer, which leads to incorporation of some Biotin-11-dUTP up to the end of the unligated Padlock probe. In addition, with synthetic short target RNA/DNA, signals may be generated by annealing the target to Padlock probe and extension to the end of the Padlock probe. Background can be avoided by leaving away the Start primer and using instead self-priming, e.g., in the case of RNA targets, after digestion of the target with various RNases, that digest the non-annealed target generating a free 3′-end for priming.

DNA affinity column: Separation using a DNA affinity column was performed essentially based on the Monarch DNA PCR and DNA cleanup kit (Monarch, NEB) whereas detection was based on the Opti-4CN N™ detection kit (Biorad). 60 microL of the 11-Biotin-dUTP labeled TN-RCA reaction was added to 420 microL of binding buffer, then loaded on spin column and centrifuged for 1 min. The column is washed 2× with 200 microL wash buffer and spun for 1 min in between. Then, 200 microL of 1/1000 dilution of Blotting grade Avidin-HPR in Antibody dilution buffer (PBST/1% BSA) from the Opti-4CN N™ Substrate Kit (Biorad) was added, centrifuged for one minute, washed 2× with 200 microL binding buffer (Monarch, NEB) (spin 1 min in between). Then, one part of Opti-4CN diluent with 9 parts H₂O was prepared and 0.2 ml of Opti-4CN substrate per 10 ml of diluents was mixed, 200 microL added to the spin column and incubated for 5-30 min with and the spin column dried with paper (spinning here will wash out the colored dye as well, but it can be restained again). In an alternative to the centrifugation step that requires a centrifuge and electricity, the filter was attached to a 10 ml syringe, and the same procedure was performed by manually generating a vacuum and sucking all the solutions into the syringe.

Lateral flow assay on Milenia Hybridetect Dipstick: 12-Fluorescein-dUTP- and 11-Biotin-dUTP-labeled samples (1-10 microL of TN-RCA reaction) were spotted on the Hybridetect Dipstick, processed as described in the manufacturers' protocol (Milenia), and photographed. Alternatively, 12-Fluorescein-dUTP labeled samples (1-10 microL of TN-RCA reaction) were spotted on the Hybridetect Dipstick, processed as described in the manufacturers protocol (Milenia) but with the addition of 1 microL of 50 microM 5′-Biotin-labeled Detection probe able to hybridize to the TN-RCA product (Biotin-5-CTCAACCTTACTACACTC-3 (SEQ ID NO: 20)) to the running buffer, and then photographed.

To determine the limits of detection of TN-RCA using the conditions outlined herein (e.g., 30° C., 30 min ligation, 2 h TN-RCA, visual detection), Zika RNA and DNA templates were serially diluted (10⁹, 10⁸, 10⁷, 10⁶, 10⁵ copies of synthetic template DNA or RNA) and TN-RCA performed and detected with either in microtiter plates, in DNA affinity columns or later flow on paper Dipsticks. The detection limits were determined to be in the range of 10⁵-10⁶ copies of input DNA or RNA, which is in the range of Zika virus reported to be present in patient samples in the acute phase (urine, blood) (10²-10⁶ PFU/ml). To reach a lower detection limit, the time of amplification can be lengthened or the sensitivity of detection is increased by enzymatic signal amplification and sensitive equipment/readers. At the detection limit, the signal was still clearly visible, whereas the background in the absence of added ligase or polymerase was negligible.

Additional methods include the following:

Digestions with Msel, Exonucleases and RNases: In various studies, TN-RCA reaction product was digested with Msel (NEB) (10 U) for 20 min at 37° C. in the presence or absence of cutting primer, Msecutprimer: 5-TTTATCTTAACTCACCAACT-3 (SEQ ID NO: 21), and the enzyme subsequently inactivated at 65° C. for 20 min. Lambda exonuclease (NEB) (0.5 U), exonuclease III (NEB) (20 U), exonuclease VIII (NEB) (2 U), T7 exonuclease (2 U), RNase H (NEB) (1 U), RNase A (Thermo Scientific) (10 U), RNase A/T1 Mix (Thermo Scientific) (1 U), RNase T1 (Invitrogen/Ambion) (1 U), ShortCut® RNase III (NEB) (0.4 U) were added to the TN-RCA reaction mixture after the ligation step was completed, or at various times during the TN-RCA reaction. In some experiments, ShortCut® RNase III (NEB) (0.4 U) was also added before the ligation step to fragment the target genomic Zika RNA. Random hexamers (Exo-Resistant Random Primer (0.2 microL of 500 microM stock) (Thermo Scientific) also was added after the ligation step.

Incorporation of dUTP and digestion with Uracil DNA glycoslyse: 1 microL of Deoxy-UTP (dUTP) from various working stock dilutions (1, 0.1, 0.01 mM) (Roche) was added to the TN-RCA reaction mixture, and Uracil-DNA glycosylase (Roche) (1 U) was added at various time points.

### Example 1

### Design of Probes for Zika Virus Detection

Stretches of 20 to 30 bp DNA sequences in which cytidine was absent were searched in aligned genomic sequences for Zika virus strains from Uganda (Genbank NC_012532.1), French Polynesia (Genbank KJ776791.1), and Brazil (Genbank KU497555.1). A stretch of 24 bp was identified in a sequence of the nonstructural protein 1 (NS1), that was C-free and identical in the three viral strains with the exception of one C in the Uganda strain. Interestingly, when the selected target sequence (5-TGTTGGTATGGAATGGAGATAAGG-3; SEQ ID NO:1) was searched using BlastN, 100% homology was found not only with Zika virus, but 95% homology was also found with Usutu virus, an emerging Flavivirus in Europe. The 5′-phosphorylated G-free padlock polynucleotide (SEQ ID NO: 4) was designed so that the 5′-end annealed to the first 12 bp of the positive strand of the target, and the 3′-end to the second 12 bp, so that ligation occurred between the two adenosines (A) in the middle. Two adenosines at the target sequence have been reported to be efficiently ligated both by T4 ligase in DNA/DNA (Nilsson et al., Nature biotechnology 18:791-793 (2000)) as well as by PBCV-1 Ligase in RNA/DNA hybrids (Lohman et al. Nucleic acids research 42:1831-1844 (2014)). The calculated Tm of the 5′-end and 3′-ends that anneal to the target sequence were both 34° C., which was relatively low due to the absence of G in the sequence. The calculated Tm of the Start primer (SEQ ID NO: 7) was 49° C. The Start primer did not reveal high homology to human DNA in BlastN searches. A search for self-dimers or hairpins in the G-free padlock probe using OligoAnalyzer 3.1 did not reveal any secondary structure that would be disadvantageous.

Due to the limited availability of Zika virus, longer pieces of Zika virus DNA targets were generated by RT-PCR as follows. Zika viral particles and RNA from Brazilian Fortaleza strain isolated from Vero cell supernatants were obtained. For generation of a Zika DNA target sequence, RT-PCR was performed as described below. Primer pairs for RT-PCR to detect Zika virus were searched using Primer3 software. Alignments of the sequences from Zika virus strains from Uganda (Genbank NC_012532.1), French Polynesia (Genbank KJ776791.1), and Brazil (Genbank KU497555.1) were further used to select primers that would amplify these three viral strains. A suitable primer pair was identified in a sequence coding for the non-structural protein 1 (NS1), with one mismatch towards the Uganda virus strain. No identical sequences were found in human DNA using BlastN searches. Alignments with other Flavivirus sequences (Dengue virus 1 to 4, Genbank KT187564.1, KT187558.1, KR296744.1, KP406806.1), Japanese Encephalitis virus (Genbank NC_001437.1), yellow fever virus (Genbank NC_002031.1), and the alpha virus Chikungunia virus (Genbank KJ451624.1 and KJ451623.1) showed that these viruses will not be amplified. RT-PCR was performed in a single tube using the Tth DNA polymerase according to the One-Step RT-PCR protocol given by the manufacturer (Roche). Briefly, 5 μL of viral RNA or particle were assembled in reaction buffer with 1 μL of 50 μM of each primer Zika forward (5-GCTTGAAATTCGGTTTGAGG-3; SEQ ID NO:8) and Zika reverse (5-CTTTCCTGGGCCTTATCTCC-3; SEQ ID NO:9). The RT reaction was at 60° C. for 30 min. Then, the samples were heated to 94° C. for 1 min, followed by 40 cycles at 94 ° C., 30 s, at 38° C., 30 s and at 72° C., 45 s, with a final elongation step at 72° C. for 7 min. The PCR products were separated by a 2% agarose gel, extracted with a gel extraction kit (Qiagen) and sequenced (Genewiz) or used for tri-nucleotide rolling circle (TN-RCA) reactions. Table 1 shows the polynucleotide used for the Zika virus assay.

TABLE 1

| | |
|---|---|
| Zika virus target sequence | TGTTGGTATGGAATGGAGATAAGG (SEQ ID NO: 1) |
| Zika virus synthetic DNA target | TAAAGATGGCTGTTGGTATGGAATGGAGATAAGGCCCAGG |
| | AAAG (SEQ ID NO: 2) |
| Zika virus synthetic RNA target | UAAAGAUGGCUGUUGGUAUGGAAUGGAGAUAAGGCCCAG |
| | GAAAG (SEQ ID NO: 3) |
| Padlock A for Zika virus | Full sequence: |
| target | pTCCATACCAACATTTTTATCTTAACTCACCAACACCATTTT |
| | TTCTAATCTCAACCTTACTACACTCTTTTTTCCTTATCTCCAT |
| | (SEQ ID NO: 4) |
| | 5′ end antisense to target: TCCATACCAACA (SEQ ID NO: 5) |
| | 3′ end antisense to target: CCTTATCTCCAT (SEQ ID NO: 6) |
| Start Primer | TGGTGTTGGTGAGTTAAG (SEQ ID NO: 7) |
| Forward primer to generate | GCTTGAAATTCGGTTTGAGG (SEQ ID NO: 8) |
| Zika target DNA by PCR | |
| Reverse primer to generate | CTTTCCTGGGCCTTATCTCC (SEQ ID NO: 9) |
| Zika target DNA by PCR | |

All sequences are written 5′-3′.

### Example 2

### Tri-Nucleotide Rolling Circle Amplification (TN-RCA) Reactions

TN-RCA Design: A scheme depicting an embodiment of the method is shown in FIG. 1. The ends of the Padlock probe oligonucleotides target specifically DNA or RNA stretches in which one or more nucleotides are missing. In addition, the base complementary to the missing base in the target sequence is absent in the TN-RCA Padlock probe, so that upon ligation, the circular template used for amplification consists only of three nucleotides. In the amplification reaction, the missing nucleotide is absent so that only correctly ligated circular templates can be amplified reducing background from mispriming and genomic DNA or RNA. Moreover, this allows incorporating labeled dNTPs (such as Fluorescein-12-dUTP, Biotin-11-dUTP, Digoxigenin-dUTP, radiolabeled dNTP, etc.) for later capture and/or optical, fluorescent, bioluminescent, chemiluminescent or enzymatic detection. dUTP is efficiently incorporated by the Phi29 polymerase, which has only a two-fold lower efficiency of incorporation when compared to dTTP and only requires p56 to replicate after Uracil excision by Uracil-DNA glycosylase. Depending on the enzymes and labels used, all reaction steps can occur at a constant temperature currently set between 20-40° C., overall assay time is estimated to be within 0.5-4 hours, depending on the steps used, the concentration of the target DNA/RNA and the equipment available (e.g. by using microfluidics or automated microtiter plates the time between the steps can be shortened).

TN-RCA reaction with Zika virus target DNA and G-free Padlock probe: 5 μL of Zika virus DNA (18 ng/reaction) and 5 μL of Hela total genomic DNA comprising all four bases as background DNA (120 ng/reaction) was denatured for 5-10 minutes with 10 μL Lysis/Denaturation solution (400 mM KOH, 5 mM EDTA), and then re-natured with 20 μL Neutralization buffer (300 mM Tris-HCL, 200 mM HCl, prepared by mixing 3 ml of 1M Tris-HCl (pH 7.5) and 2 ml of 1 M HCl with 5 ml of water). Then, 10 μL of neutralized DNA was immediately added to the Ligation reaction mixture containing 3 μL 10× T4 Ligase buffer (NEB), 6 μL 1 μM 5′-phosphorylated G-free Padlock probe (Sigma), 2 μL T4 Ligase (NEB), and to 30 μL ultrapure distilled water, and then incubated at 30° C. for 30 minutes. To this solution, a TN-RCA polymerization mixture was added consisting of 6 μL Phi29 10× reaction buffer (NEB), 1.2 μL of 50 μM Start primer (Sigma), 0.6 μL of 10 μg/μL BSA (NEB), 1.2 μL dNTP mix (dATP, dGTP, dTTP, 25 mM each), labeled Fluorescein-12-dUTP (various amounts of 1 mM stock), and/or Biotin-11-dUTP (various amounts of 1 mM stock), and 1 μL 2000 U/ml Phi29 Polymerase (NEB), various amounts of distilled water to 60 μL, and then incubated at 30° C. for 0.5 to 6 hours. At the end, 10 μL of 6× Gel loading dye was added, and the sample was mixed and separated by 1.5% Agarose Gel electrophoresis. The gel was photographed under UV light using a Sony Cybershot 13.6 megapixel DSC-W300 camera, and the three channels (visible, red, green) visualized using Adobe Photoshop CS 5.1 software. When Biotin-11-dUTP was used as a label, biotinylated single stranded amplification products were immobilized by binding to neutravidin-coated microtiter plates (Pierce) or by immobilization on DNA binding columns and washed. The immobilized amplification products were then bound with Avidin-coupled horseradish peroxidase (Avidin-HRP), washed, and detected by the formation of a colored insoluble Opti-4CN precipitate upon reaction with Avidin-HRP.

There was specific amplification (fluorescein-12-dUTP labeled product) in the presence of Zika virus DNA and T4 Ligase when observed in the green channel (520 nm), but no specific amplification in the presence of Zika virus without T4 Ligase or in the presence of HeLa DNA only, with or without T4 ligase, when observed in the green channel. In the absence of ethidium bromide in the agarose gel, specific amplification was observed in all channels. There was specific amplification (biotinylated product) in the presence of Zika virus DNA and T4 Ligase, but not in the presence of Zika virus DNA without T4 Ligase or in the presence of HeLa DNA alone, without or without T4 Ligase.

In a related study, a Zika PCR DNA fragment containing the TN target sequence was generated, spiked or not spiked into Hela cell genomic DNA as non-specific human background, denatured/renatured, and TN-RCA performed with and without T4 DNA ligase and in the presence of 420 microM dNTP (without dCTP) and 16 microM Fluorescein-12-dUTP. After separation of the TN-RCA products in a 1.5% agarose gel containing low amounts of Gel Red, a stronger signal was detected after UV illumination in lanes representing TN-RCA product and background genomic DNA. When observed in the red channel, signals were observed in all lanes. However, when observed in the green channel, only the specifically fluorescently labeled TN-RCA product derived from amplification of Zika virus DNA was detected.

Amplification for shorter times (1 h) with increased amounts of Fluorescein-dUTP (5× or 80 microM) still resulted in a TN-RCA product specifically detectable only in the green channel, albeit 16 microM dNTP was found to be already limiting, so that in the following experiments 80 microM dNTP was used. Lowering dNTP was possible also because almost none are used for other reactions (Km and Kd of Phi29 for unmodified dNTP are 0.5 microM (NEB) and 1.4 microM, respectively). In the complete absence of dTTP even more Fluorescein-12-dUTP may be incorporated; however, amplification to high molecular weight TN-RCA products appears to be less efficient and quenching of fluorescence may occur when too dense. For consistency, all these and subsequent experiments were performed at 30° C.; however, TN-RCA tested at ambient temperature (22° C.) was fully functional albeit slightly less efficient as detected in agarose gels. For amplification at ambient temperatures, other polymerases such as IsoPol™ DNA Polymerase (ArcticZymes) may also be advantageous. Since detection of fluorescent TN-RCA products do not require additional detection by fluorescent dyes in the agarose gel (Gel Red, Ethidium bromide), subsequent experiments were separated by agarose gels in the absence of these dyes.

TN-RCA reaction with Zika virus RNA and G-free Padlock probe: 5 μL of Zika virus RNA synthetic template (18 ng/reaction) or Zika virus RNA (isolated from heat inactivated supernatant of infected Vero cells using RNAease virus RNA extraction kit (Qiagen)) and 5 μL of Hela total genomic DNA comprising all four bases as background DNA (120 ng/reaction) was denatured for 5-10 minutes with 10 μL Lysis/Denaturation solution (400 mM KOH, 5 mM EDTA), and then re-natured with 20 μL Neutralization buffer (300 mM Tris-HCL, 200 mM HCl, prepared by mixing 3 ml of 1M Tris-HCl (pH 7.5) and 2 ml of 1 M HCl with 5 ml of water). Then, 10 μL of neutralized DNA was immediately added to the Ligation reaction mixture containing 3 μL 10× PBCV-1 Ligase buffer (NEB), 6 μL 1 μM 5′-phosphorylated G-free Padlock probe (Sigma), 2 μL PBCV-1 Ligase (NEB), and to 30 μL ultrapure distilled water, and then incubated at 30° C. for 30 minutes. To this solution, a TN-RCA polymerization mixture was added consisting of 6 μL Phi29 10× reaction buffer (NEB), 1.2 μL of 50 μM start primer (Sigma), 0.6 μL of 10 μg/μL BSA (NEB), 1.2 μL dNTP mix (dATP, dGTP, dTTP, 25 mM each), labeled Fluorescein-12-dUTP (various amounts of 1 mM stock), and/or Biotin-11-dUTP (various amounts of 1 mM stock), and 1 μL 2000 U/ml Phi29 Polymerase (NEB), various amounts of distilled water to 60 μL, and then incubated at 30° C. for 0.5- to 6 hours. At the end, 10 μL of 6× Gel loading dye was added, and the sample was mixed and separated by 1.5% Agarose Gel electrophoresis. The gel was photographed under UV light using a Sony Cybershot 13.6 megapixel DSC-W300 camera, and the three channels (visible, red, green) visualized using Adobe Photoshop CS 5.1 software. When Biotin-11-dUTP was used as a label, biotinylated single stranded amplification products were immobilized by binding to neutravidin-coated microtiter plates (Pierce) or by immobilization on DNA binding columns and washed. The immobilized amplification products were then bound with Avidin-coupled horseradish peroxidase (Avidin-HRP), washed, and detected by the formation of a colored insoluble Opti-4CN precipitate upon reaction with Avidin-HRP.

There was specific amplification (fluorescein-12-dUTP labeled product) in the presence of Zika virus RNA and T4 Ligase when observed in the green channel (520 nm), but no specific amplification in the presence of Zika virus without PBCV-1 Ligase or in the presence of HeLa DNA only, with or without PBCV-1 ligase, when observed in the green channel. In the absence of ethidium bromide in the agarose gel, specific amplification was observed in all channels. There was specific amplification (biotinylated product) in the presence of Zika virus RNA and PBCV-1 Ligase, but not in the presence of Zika virus RNA without PBCV-1 Ligase or in the presence of HeLa DNA alone, without or without PBCV-1 Ligase.

Since Phi29 polymerase has 3′ to 5′ RNase activity which digests ssRNA, the remaining non-digested RNA annealed to the ligated padlock can serve as the start site of TN-RCA so that the start primer is not necessary. Moreover, to ensure a 3′-end close to the target sequence, ssDNA can be cleaved by DNA Glycosylase and Endonuclease IV. Accordingly, with DNA, self-priming can be initiated after digesting the non-annealed DNA by the 3′ to 5′ exonuclease activity of Phi29.

In a related study, a set of available RNases was tested to cleave the target RNA before or during the TN-RCA reaction so that self-priming in the absence of the Start primer can occur more efficiently. The Padlock probe length was also altered from 84 bp to 74 bp (equivalent to about 6 helical turns of B-DNA), Padlock probes were used that anneal to their target with one or two mismatches (to facilitate cleavage by RNases). The sequences of the Padlock probes were as follows (bold: ends that anneal to the target sequence):

Padlock probe 74 bp (G-free):
(SEQ ID NO: 22)
5-p-
TCCATACCAACATTTTTATCTTAACTCACCAACACCATCTCAACCTTACT
ACACTCTTTTTTCCTTATCTCCAT-3
Padlock probe 74 bp/one mismatch (G-free;
mismatched bases is lower case):
(SEQ ID NO: 23)
5-p-
TCCATACCAACATTTTTATCTTAACTCACCAACACCATCTCAACCTTACT
ACACTCTTTTTTCCcTATCTCCAT-3
Padlock probe 74 bp/two mismatch (G-free;
mismatched bases are lower case):
(SEQ ID NO: 24)
5-p-
TCCATACCAtCATTTTTATCTTAACTCACCAACACCATCTCAACCTTACT
ACACTCTTTTTTCCcTATCTCCAT-3.

The shortened Padlock probes with and without mismatches improved detection of the target RNA. The presence of a mismatch and the addition of an RNase such as RNase H and/or RNase A and/or RNase III not only specifically hydrolyzes the dsRNA and the RNA in RNA/DNA hybrids, thus facilitating self-priming, but also facilitates the release of the circular Padlock probe from the intertwined RNA.

To generate shorter TN-RCA reaction products for certain applications, ddNTP can be added during the amplification. Another method for TN-RCA product fragmentation is by using Uracil DNA glycosylase (UDG) and endonuclease IV which cuts the abasic sites. UDG and endonuclease IV were not able to digest the TN-RCA reaction products with incorporated Fluorescein-12-dUTP or Biotin-11-dUTP. However, UDG alone and in combination of endonuclease IV efficiently fragmented the TN-RCA reaction products containing various amounts of dUTP. The UDG fragmented reaction products could initiate TN-RCA with a circularized Padlock probe suggesting that the UDG-digested fragments can serve as targets able for self-priming leading to overall increased TN-RCA. It can be expected that the many modifications described for the RCA method can also be used to increase the sensitivity and specificity of the TN-RCA method.

In situ TN-RCA: The oligonucleotide containing the Zika virus target sequence (ZTargetamine: 5-amine-C6-MMT-TAAAGATGGCTGTTGGTATGGAATGGAGATAAGGCCCAGGAAAG (SEQ ID NO: 2)-3) was covalently attached to an IgG secondary antibody (anti-rabbit IgG, whole molecule, produced in goat (Sigma)), using an oligonucleotide-conjugation kit according to the manufacturers' procedure (Abcam). Hela cells were grown in Falcon 8-well cell culture slides, fixed with 10% Formalin/PBS, permeabalized with Saponin and incubated with primary (anti-Akt1/2/3 (Santa Cruz)) and oligonucleotide-labeled secondary antibody using standard protocols as for immunofluorescence staining, and TN-RCA performed in situ essentially as described above, washed with PBS, and photographed with a fluorescent microscope (BZ-X710, Keyence). Target was visualized allowing detection of the oligonucleotide-labeled antibody.

Additional observations: To examine the ability to detect intact Zika virus RNA, Zika-virus genomic RNA was isolated from supernatants of Zika-virus infected Vero cells. When compared to short synthetic Zika virus target RNA, using Zika-virus genomic RNA gave less efficient TN-RCA amplification, even when amounts and conditions were varied. When exonucleases were added, a robust amplification was observed even with genomic Zika virus RNA. Further studies using exonucleases revealed that the increase in TN-RCA amplification was associated with increased self-ligation of the Padlock probe in the absence of target DNA/RNA; thus, exonucleases may be associated with a decreased specificity in this assay. In contrast, short target oligonucleotides covalently attached to antibodies efficiently amplified by self-priming and were detectable by fluorescent microscopy; thus, TN-RCA is useful for detection of specific cellular targets such as DNA/RNA directly or attached to proteins/antibodies, possibly with lower background.

In various aspects of the disclosure a start primer is not used, and self-priming by the target DNA/RNA is employed to initiate the amplification reaction. Whereas self-priming is easy achieved with short synthetic DNA/RNA targets, longer genomic dsRNA is preferably fragmented to generate a free 3′ end. The intrinsic 3′ to 5′ RNase activity of Phi29 polymerase can digest the RNA that is not hybridized to the target sequence, generating 3′-ends that can be used as starting point for self-priming in the absence of Start primer. The digestion of RNA by Phi29 is facilitated by RNase III which cleaves dsRNA often formed in longer RNA targets. A set of available RNases was tested to cleave the target RNA before or during the TN-RCA reaction so that self-priming in the absence of the start primer can occur. Padlock probe length also was changed from 84 bp to 74 bp (equivalent to about 6 helical turns of B-DNA), and Padlock probes that anneal to their target with one or two mismatches (to facilitate cleavage by RNases) were used. The presence of a mismatch and the addition of an RNase, such as RNase H and/or RNase A and/or RNase III, not only specifically hydrolyzes the dsRNA and the RNA in RNA/DNA hybrids, thus facilitating self-priming, but also release the circular Padlock probe form the intertwined RNA.

Two-step TN-RCA: To facilitate detection with lateral flow assay and to generate templates able to serve as targets in a second amplification step, several methods were used to fragment the fluorescein- and/or Biotin-labeled TN-RCA reaction products after or during the assay. In these experiments, circularized Padlock probes (cLPadlocks) were used to evaluate whether the fragments generated in a first TN-RCA amplification can serve as targets and primers for a second TN-RCA amplification. It was found that digestion with a restriction enzyme (Msel) was feasible but required the addition of the complementary G-free oligonucleotide containing the restriction site; the addition of a circularized Padlock probe weakly increased amplification, suggesting that the digested, partially double-stranded fragments did not efficiently serve as target for a second TN-RCA reaction without additional treatment.

It was also tested whether the incorporation of Fluorescein-12-dUTP makes the TN-RCA reaction product sensitive to digestion by Uracil glycosylase (UDG) and endonuclease IV, which was not the case. Therefore, dUTP was added to the TN-RCA reaction mixture at various concentrations and time points in the presence and absence of UDG and endonuclease IV and the reaction products separated by agarose gel; the UDG alone and in combination of endonuclease IV efficiently fragmented the TN-RCA reaction products. However, the presence of endonuclease IV did not much enhance fragmentation by UDG when compared to UDG alone. Unexpectedly, in the presence of circularized Padlock probes, the presence of endonuclease IV gave some background signals assumed to be the result of acting as protein-mediated starting point for Phi29, so that in subsequent experiments endonuclease IV was not used.

The UDG fragmented reaction products from the first unlabeled TN-RCA reaction did not serve as very efficient targets for a second complete TN-RCA reaction without further treatments, possibly because the ligation reaction is not efficient with targets containing dUTP and abasic sites after UDG digestion; however, the addition of a circularized Padlock probe to the reaction increased TN-RCA amplification with UDG-digested TN-RCA suggesting that the UDG-digested fragments can serve as targets and enable self-priming. Secondary TN-RCA amplification was increased by the addition of circularized Padlock probes to UDG-digested TN-RCA products from genomic Zika RNA, suggesting that the fragments generated in the presence of dUTP and UDG could serve as primers for a secondary TN-RCA reaction.

Instead of two-step TN-RCA, the generation of labeled products can also be increased by adding Exo resistant random hexamer oligonucleotides which anneal to the TN-RCA reaction product and incorporate F12-dUTP during the TN-RCA reaction. However, since random hexamers and the products generated by random hexamer would anneal to pre-circularized Padlock probes and efficiently start TN-RCA, un-ligated complementary G- and C-free Padlock probe are preferred in a secondary amplification step. However, the addition of random hexanucleotides to the reaction mixture appeared to increase the efficiency of TN-RCA by facilitating priming and incorporating more labels, and genomic Zika virus RNA could be detected by TN-RCA and lateral flow assay with Dipsticks in about the same time (3 h) as with conventional RT-PCR and agarose gel.

### Example 3

### Design of Probes for HPV Detection and Detection of Target DNA

HPV General Primer 6 plus (GP6plus: 5- GAAAAATAAACTGTAAATCATATTC-3; SEQ ID NO:15) which allows amplifying 14 high risk HPV subtypes contains only 2 G, and alignment of these HPV virus sequences revealed that the region around GP6plus has a low number of G in all 14 HPV virus. TN-RCA reactions were conducted as described in Example 2 using the polynucleotides for HPV shown in Table 2.

There was specific amplification (fluorescein-12-dUTP labeled or biotinylated product) in the presence of HPV DNA and T4 Ligase, but not in the absence of ligase or for background DNA. In this case, the G-free padlock probe annealed to the HPV target sequence containing one C. Mis-matched annealing of such padlock probes may need lower temperatures or modified buffer conditions, and their ligation occurs as long as the bases at the ligation junction are not mismatched. The incorporation of “Universal base analogues” into the Padlock sequence opposite the C in the target sequence may help in annealing while still allowing TN-RCA in the presence of only three dNTP.

TABLE 2

| | |
|---|---|
| HPV DNA target sequence | GAGGAATATGATTTACAGTTTATTTTTGA |
| (present in HPV16 and HPV45) | (SEQ ID NO: 10) |
| Padlock for HPV target | Full sequence: |
| | pCATATTCCTCTTTTTATCTTAACTCACCAACACCATTT |
| | TTTCTAATCTCAACCTTACTACACTCTTTTTTTCAAAAA |
| | TAAACTCTAAAT (SEQ ID NO: 11) |
| | 5′ end antisense to target: CATATTCCTC (SEQ ID NO: 12) |
| | 3′ end antisense to target: TCAAAAATAAACTCTAAAT (SEQ |
| | ID NO: 13) |
| Start Primer | TGGTGTTGGTGAGTTAAG (SEQ ID NO: 7) |
| HPV forward universal primer to | TTTGTTACTGTGGTAGATACTAC (SEQ ID NO: 14) |
| generate target DNA by PCR | |
| HPV reverse universal primer | GAAAAATAAACTGTAAATCATATTC (SEQ ID NO: 15) |
| GP6+ to generate target DNA by PCR | |

All sequences are written 5′-3′.

### Example 4

### Design of Probes for Norovirus Detection and Detection of Target RNA

The Norovirus GII sequence was screened for stretches of C-free DNA sequences and candidate sequences checked for uniqueness using NBlast searches. TN-RCA reactions were conducted as described in Example 2 using the polynucleotides for Norovirus shown in Table 3, and specific amplification was observed in the presence of Norovirus RNA and T4 Ligase.

TABLE 3

| | |
|---|---|
| Norovirus RNA target sequence | GAGUUGGUAUGAAUAUGAAUGAGGA (SEQ ID NO: 16) |
| Padlock for Norovirus target | Full sequence: |
| | pTCATACCAACTCTTTTTATCTTAACTCACCAACACCAT |
| | CTCAACCTTACTACACTCTTTTTTCCTCATTCATAT |
| | (SEQ ID NO: 17) |
| | 5′ end antisense to target: TCATACCAACTC (SEQ ID NO: 18) |
| | 3′ end antisense to target: CCTCATTCATAT (SEQ ID NO: 19) |
| Start Primer | TGGTGTTGGTGAGTTAAG (SEQ ID NO: 7) |

All sequences are written 5′-3′.

### Discussion

TN-RCA is a novel isothermal amplification technique allowing sensitive detection, quantification and diagnosis of any natural or synthetic DNA or RNA containing short stretches of sequences with only three of the four nucleotides, with low background and in short time. All the reactions and detection can be performed in liquid form, permitting TN-RCA to be used in the context of microfluidics or automated microtiter-based platform. TN-RCA also is suitable for in situ detection, quantification and localization of DNA or RNA (and of specific point mutations) in tissue sections, including frozen and paraffin-embedded tissue sections, in fixed cells, as well as in dried samples. TN-RCA also is suitable for use in point of care tests (POCT), as clinical, laboratory or field kits, or as part of laboratory techniques for in vitro and in situ measurement of specific DNA or RNA. Moreover, TN-RCA is suitable for detecting and identifying/authenticating other molecules (e.g., antibodies, proteins, lipids, nucleic acids, organisms/GMO, chemicals, solutions, such as color in paintings or biometric ink in writings or microdots), and other objects of interest that have been tagged or spiked with oligonucleotides (e.g., stabilized with phosphothionate linkage) encoding the complementary target sequence.

TN-RCA has several advantages over RCA. For example, TN-RCA has increased specificity of amplification and lower background. Due to the presence of only three dNTPs in the reaction, only correctly ligated, circular G-free Padlock probes will amplify and incorporate labeled dNTPs into the TN-RCA product, which increases the specific signal and lowers the signals from background amplification from endogenous RNA/DNA. High molecular weight DNA/RNA and their complexes with proteins that often co-migrate with the RCA product and give background is not detected, since Fluorescein-12-dUTP is only incorporated after specific amplification into TN-RCA products. Moreover, since all polymerase molecules and dNTPs are used only for specific amplification, they are not consumed in non-specific incorporations, thus enhancing the specific incorporation at lower concentrations. In RCA, background signals are generated from the presence of high molecular weight genomic DNA, from amplification due to presence of nicks in genomic DNA or RNA, or from non-specific annealing and/or amplification of probe, Start primer, or labeled Detection probe to genomic DNA or RNA.

Another advantage of TN-RCA is increased sensitivity, higher speed, and lower cost. For detection, when compared to labeled detection probes that usually contain one label per probe (and thus one label per detected concatemeric repeat in the RCA product), TN-RCA can incorporate many and multiple-type labeled dNTP (e.g., the G-free Padlock probe used has 22 A to incorporate labeled dUTP). Since the label is incorporated during the TN-RCA, less time is required for detection since no hybridization is required, and no specific detection probes need to be synthesized, lowering the assay cost.

Yet another advantage of TN-RCA is maintenance of specificity at lower reaction temperatures. Due to the absence of, e.g., guanosine in the G-free Padlock probe, or alternatively due to the absence of cytidine in the C-free Padlock probe (both nucleotides forming triple-bonds with high affinity in DNA and RNA), the overall melting temperature is lower with consequent lower secondary structure of Padlock probe and target sequence, as well as lower self-priming and self-annealing features of the Padlock probe. Similarly, the Start primer has a lower secondary structure and self-dimer and self-priming features, again reducing background. These features allow annealing the ends of the Padlock probes at lower temperature (e.g., isothermally at 20-37° C.) and/or the use of longer Padlock probe ends for annealing to the target sequences, which leads to a higher specificity and sensitivity. Moreover, since stretches of 20-30 bp or longer target sequences lacking a particular nucleotide are rare in the genome (due to the overall equal presence of all four nucleotides and their coding biased distribution), the specificity of TN-RCA is higher, although it requires the presence of such TN stretches in the target sequences.

The foregoing Examples demonstrate that the methods and kits described herein allow for rapid and specific detection of a target polynucleotide sequence in a sample. The design strategy and protocol provided above is applicable to pathogens of interest including, but not limited to, the pathogens described herein, as well as synthetic target polynucleotides.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

What is claimed:

1. A method for detecting a target polynucleotide sequence in a sample comprising:
(1) a circularization step comprising combining the target polynucleotide sequence with (a) a padlock probe polynucleotide sequence comprising a 5′ end complementary to a first section of the target polynucleotide sequence and a 3′ end complementary to a second section of the target polynucleotide sequence adjacent to the first section, wherein the target polynucleotide sequence lacks a missing base selected from adenine, cytosine, guanine, thymine, uracil, and combinations thereof, and wherein the padlock probe polynucleotide sequence lacks a base complementary to said missing base; and (b) a ligase, to form a circular padlock probe;
(2) an amplification step comprising combining the circular padlock probe with (a) a polymerase, and (b) a mixture of deoxynucleotide triphosphates (dNTPs), wherein the mixture of dNTPs lacks said missing base, and optionally (c) a start primer comprising a polynucleotide sequence complementary to a portion of the padlock probe polynucleotide sequence, to form antisense copies of the padlock probe; and
(3) a detection step comprising identifying the antisense copies of the padlock probe.

2. The method of claim 1, wherein said missing base is adenine and the mixture of dNTPs lacks dATP.

3. The method of claim 1, wherein said missing base is cytosine, and the mixture of dNTPs lacks dCTP.

4. The method of claim 1, wherein said missing base is guanine, and the mixture of dNTPs lacks dGTP.

5. The method of claim 1, wherein said missing base is thymine, and the mixture of dNTPs lacks dTTP.

6. The method of claim 1, wherein said missing base is uracil, and the mixture of dNTPs lacks dUTP and dTTP.

7. The method of any of claims 1-6, wherein the target polynucleotide sequence is about 20 to about 40 nucleotides in length.

8. The method of any of claims 1-7, wherein the padlock probe polynucleotide sequence is about 50 to about 200 nucleotides in length.

9. The method of any of claims 1-8, wherein the target polynucleotide sequence is a naturally occurring DNA or RNA sequence.

10. The method of any of claims 1-8, wherein the target polynucleotide sequence is a synthetic polynucleotide.

11. The method of any of claims 1-10, wherein the target polynucleotide sequence is a pathogen polynucleotide sequence.

12. The method of claim 11, wherein the pathogen is a flavivirus, optionally Zika virus; human papillomavirus; Chlamydia tracomatis; Norovirus; or Neisseria gonorrhoeae.

13. The method of claim 1, wherein the target polynucleotide sequence comprises a sequence selected from SEQ ID NOs: 1, 2, 3, and 10, and variants having at least 90% sequence identity to any of the foregoing.

14. The method of claim 1, wherein the padlock probe polynucleotide sequence comprises a sequence selected from SEQ ID NOs: 4 and 11, and variants having at least 90% sequence identity to any of the foregoing.

15. The method of claim 1, comprising a start primer comprising a polynucleotide sequence set forth in SEQ ID NO: 7 and variants having at least 90% sequence identity to SEQ ID NO: 7.

16. The method of any of claims 1-15, wherein steps (1) and (2) are performed in a single reaction vessel.

17. The method of any of claims 1-16, wherein steps (1) and (2) are performed at a temperature between about 20° C. to about 40° C.

18. The method of any of claims 1-17, wherein the ligase is T4 ligase or PBCV-1 ligase.

19. The method of any of claims 1-19, wherein the mixture of dNTPs comprises labeled dNTPs, optionally selected from the group consisting of fluorescent dNTPs, biotinylated dNTPs, digoxigeninated dNTPs, radiolabeled dNTPs, and combinations thereof.

20. The method of claim 1, wherein the mixture of dNTPs comprises dUTP, and the method further comprises, prior to the detection step (3), cleaving the product of amplification step (2) with Uracil-DNA-glycosylase to generate single-stranded amplification products, and repeating amplification step (2).

21. A kit for detection of a pathogen polynucleotide sequence in a sample comprising:
(1) a padlock probe polynucleotide sequence comprising a 5′ end complementary to a first section of the pathogen polynucleotide sequence and a 3′ end complementary to a second section of the pathogen polynucleotide sequence located adjacent to the first section; wherein the pathogen polynucleotide sequence lacks a missing base selected from adenine, cytosine, guanine, thymine, uracil, and combinations thereof; and wherein the padlock probe polynucleotide sequence lacks a base complementary to said missing base; (2) a ligase that anneals the 5′ and 3′ ends of the padlock probe polynucleotide together to form a circular padlock probe; (3) a polymerase; (4) a mixture of deoxynucleotide triphosphates (dNTPs) wherein the mixture of dNTPs lacks said missing base; and optionally (5) a start primer comprising a polynucleotide sequence complementary to a portion of the padlock probe polynucleotide sequence.

22. The kit of claim 20, wherein (1) said missing base is adenine and the mixture of dNTPs lacks dATP; (2) said missing base is cytosine and the mixture of dNTPs lacks dCTP; (3) said missing base is guanine, and the mixture of dNTPs lacks dGTP; (4) said missing base is thymine and the mixture of dNTPs does not comprise dTTP; or (5) said missing base is uracil and the mixture of dNTPs lacks dUTP and dTTP.

23. The kit of claim 20 or 21, wherein the pathogen is Zika virus; the padlock probe comprises a polynucleotide sequence selected from the group consisting of SEQ ID NOs: 1-3, and variants having at least 90% sequence identity to any of the foregoing; and the start primer comprises a polynucleotide sequence set forth in SEQ ID NO: 7, and variants having at least 90% sequence identity to any of the foregoing.

24. The kit of claim 20 or 21, wherein the pathogen is human papillomavirus; the padlock probe comprises a polynucleotide sequence set forth in SEQ ID NO: 10, and variants having at least 90% sequence identity to SEQ ID NO: 10; and the start primer comprises a polynucleotide sequence set forth in SEQ ID NO: 7, and variants having at least 90% sequence identity to any of the foregoing.

25. The kit of any of claims 20-23, wherein the mixture of dNTPs comprises labeled dNTPs, optionally selected from the group consisting of a fluorescent dNTPs, biotinylated dNTPs, digoxigeninated dNTPs, radiolabeled dNTPs, and combinations thereof.


SEQUENCE LISTING (24 sequences)

SEQ ID NO: 1 - DNA, 24 bp, Artificial Sequence
  Synthetic Polynucleotide
  misc_feature: Zika virus target sequence
tgttggtatggaatggagataagg

SEQ ID NO: 2 - DNA, 44 bp, Artificial Sequence
  Synthetic polynucleotide
  misc_feature: Zika virus synthetic DNA target
taaagatggctgttggtatggaatggagataaggcccaggaaag

SEQ ID NO: 3 - DNA, 19 bp, Artificial Sequence
  Synthetic polynucleotide
  misc_feature: Zika virus synthetic RNA target
tttcctgggccttatctcc

SEQ ID NO: 4 - DNA, 84 bp, Artificial Sequence
  Synthetic polynucleotide
  misc_feature: Padlock A for Zika virus target
tccataccaacatttttatcttaactcaccaacaccattttttctaatctcaaccttactacactcttttttccttatctccat

SEQ ID NO: 5 - DNA, 12 bp, Artificial Sequence
  Synthetic polynucleotide
  misc_feature: 5′ end antisense to target
tccataccaaca

SEQ ID NO: 6 - DNA, 12 bp, Artificial Sequence
  Synthetic polynucleotide
  misc_feature: 3′ end antisense to target
ccttatctccat

SEQ ID NO: 7 - DNA, 18 bp, Artificial Sequence
  Synthetic polynucleotide
  misc_feature: Start Primer
tggtgttggtgagttaag

SEQ ID NO: 8 - DNA, 20 bp, Artificial Sequence
  Synthetic polynucleotide
  misc_feature: Forward primer to generate Zika target DNA by PCR
gcttgaaattcggtttgagg

SEQ ID NO: 9 - DNA, 20 bp, Artificial Sequence
  Synthetic polynucleotide
  misc_feature: Reverse primer to generate Zika target DNA by PCR
ctttcctgggccttatctcc

SEQ ID NO: 10 - DNA, 29 bp, Artificial Sequence
  Synthetic polynucleotide
  misc_feature: HPV DNA target  sequence
gaggaatatgatttacagtttatttttga

SEQ ID NO: 11 - DNA, 89 bp, Artificial Sequence
  Synthetic polynucleotide
  misc_feature: Padlock for HPV target- Full Sequence
catattcctctttttatcttaactcaccaacaccattttttctaatctcaaccttactacactctttttttcaaaaataaactctaaat

SEQ ID NO: 12 - DNA, 10 bp, Artificial Sequence
  Synthetic polynucleotide
  misc_feature: 5′ end antisense to target
catattcctc

SEQ ID NO: 13 - DNA, 19 bp, Artificial Sequence
  Synthetic polynucleotide
  misc_feature: 3′ end antisense to target
tcaaaaataaactctaaat

SEQ ID NO: 14 - DNA, 23 bp, Artificial Sequence
  Synthetic polynucleotide
  misc_feature: HPV forward universal primer to generate target DNA by PCR
tttgttactgtggtagatactac

SEQ ID NO: 15 - DNA, 25 bp, Artificial Sequence
  Synthetic polynucleotide
  misc_feature: HPV reverse universal primer GP6+ to generate target DNA by PCR
gaaaaataaactgtaaatcatattc

SEQ ID NO: 16 - DNA, 25 bp, Artificial Sequence
  Synthetic polynucleotide
  misc_feature: Norovirus RNA target sequence
gaguugguaugaauaugaaugagga

SEQ ID NO: 17 - DNA, 74 bp, Artificial Sequence
  Synthetic polynucleotide
  misc_feature: Padlock for Norovirus target- Full Sequence
tcataccaactctttttatcttaactcaccaacaccatctcaaccttactacactcttttttcctcattcatat

SEQ ID NO: 18 - DNA, 12 bp, Artificial Sequence
  Synthetic polynucleotide
  misc_feature: 5′ end antisense to target
tcataccaactc

SEQ ID NO: 19 - DNA, 12 bp, Artificial Sequence
  Synthetic polynucleotide
  misc_feature: 3′ end antisense to target
cctcattcatat

SEQ ID NO: 20 - DNA, 18 bp, Artificial Sequence
  Synthetic Polynucleotide
  misc_feature: Biotin
ctcaaccttactacactc

SEQ ID NO: 21 - DNA, 18 bp, Artificial Sequence
  Synthetic Polynucleotide
  misc_feature: Msecutprimer
ctcaaccttactacactc

SEQ ID NO: 22 - DNA, 74 bp, Artificial Sequence
  Synthetic Polynucleotide
  misc_feature: Padlock probe
tccataccaacatttttatcttaactcaccaacaccatctcaaccttactacactcttttttccttatctccat

SEQ ID NO: 23 - DNA, 74 bp, Artificial Sequence
  Synthetic Polynucleotide
  misc_feature: Padlock Probe
tccataccaacatttttatcttaactcaccaacaccatctcaaccttactacactcttttttccctatctccat

SEQ ID NO: 24 - DNA, 74 bp, Artificial Sequence
  Synthetic Polynucleotide
  misc_feature: Padlock probe
tccataccatcatttttatcttaactcaccaacaccatctcaaccttactacactcttttttccctatctccat